(19) United States
(12) Patent Application Publication
(10) Pub. No.: US 2024/0188939 A1
(43) Pub. Date: Jun. 13, 2024

(54) Sample Collection Devices, Systems, and Methods

(71) Applicant: 3M INNOVATIVE PROPERTIES COMPANY, St. Paul, MN (US)

(72) Inventors: Audrey A. Sherman, Woodbury, MN (US); Brett J. Sitter, Marine On St. Croix, MN (US); Alan R. Dombrowski, Woodbury, MN (US); Laura R. Nereng, Woodbury, MN (US); Michael R. Berrigan, Oakdale, MN (US); Narina Y. Stepanova, Woodbury, MN (US); Adam S. Troness, Dellwood, MN (US); Michael C. Wohl, Bethesda, MD (US)

(21) Appl. No.: 18/553,770

(22) PCT Filed: Feb. 11, 2022
(86) PCT No.: PCT/IB2022/051252

§ 371 Date: Oct. 3, 2023

Related U.S. Application Data
(60) Provisional application No. 63/200,958, filed on Apr. 6, 2021.
(60) Provisional application No. 63/203,831, filed on Aug. 2, 2021.

(51) Int. Cl. A61B 10/00; A61B 5/08

(52) U.S. Cl. CPC A61B 10/00; A61B 5/082; A61B 2010/0087

(57) ABSTRACT

The present disclosure relates to sample collection devices and sample collection and testing devices. The devices described herein includes a housing defining a fluid channel from a first portion to a second portion. The devices further include a porous sample collection media disposed within the housing and in fluid communication with the fluid channel. The devices further include a fluid inlet port disposed in fluid communication with the porous sample collection media. The fluid inlet port is configured to direct a test fluid onto the porous sample collection media. The sample collection device further includes an assay configured to receive a fluid that was incident on the porous sample collection media (and eluent) and test that eluent for the presence (or absence) of a pathogen or virus.

### TECHNICAL FIELD

The present disclosure relates generally to sample collection devices and systems, methods for using the sample collection devices and systems, and sample collection and testing devices and systems.

### BACKGROUND

As Covid-19 has reached pandemic status, increased availability of diagnostic testing is important to help identify and control the serious illness. This illness has highlighted the need for widespread availability of such diagnostic tests even after the pandemic ends. Diagnostic tests used to test for the presence of a virus or other pathogen in the airways, throat, or nasopharynx typically involve the insertion of a swab into the back of the nasal passage, the mid-turbinate area of the nasal passage, the anterior nares, or the throat to obtain a sample. The swab is then inserted into a container and analyzed or sent to a lab for processing. Other diagnostic tests involve collecting a saliva sample and then placing it in a container. Currently available at-home viral tests (e.g., COVID-19 tests) involve a nasal swab and a test kit (for example, the Ellume™ test, the Abbot™ BinaxNOW™ test, and the Lucira™ All-in-One test kit). Tests that utilize nasal swab samples or saliva contend with contaminants that can interfere with the various diagnostic tests. As a result, these sample types require a purification step when using RT-PCR molecular testing.

### SUMMARY

The inventors of the present disclosure recognized that the sample collection devices and test processes described above have various challenges. For example, most of the available tests require that the collection device be processed at a laboratory, increasing cost and delaying delivery of results. Further, many of the test methods require that the sample collection mechanism be a nasopharyngeal or other type of nasal or oral swab, which is uncomfortable for the user. This discomfort can cause users to opt out of testing. Further, there may be possibility of contamination of the sample during transfer to the clean container, removal from the container, etc. Due to the multiple steps and devices involved and the possibility of contamination of the sample, such conventional methods and devices for sample collection and eluent testing may be used by only trained professionals (e.g., medical personnel), and may be complicated for use by a user with little or no training.

As such, the inventors of the present disclosure recognized that there is a need for an inexpensive, simple to use, and reliable sample collection system that may be used (even by laypeople) to obtain a sample for testing for the presence of a target virus, target pathogen, or other target analyte, in a collected sample. The inventors of the present disclosure sought to create easy-to-use, inexpensive integrated sample collection and testing devices in which sample collection and sample testing happen in a single integrated device that can be used in any location by a layperson. Additionally, the inventors sought to create an integrated sample collection and testing device that did not require the user to undergo a nasopharyngeal or other nasal or oral swab.

Thus, the inventors of the present disclosure invented the sample collection and testing devices and methods described herein. The sample collection and testing devices described herein are capable of collecting an aerosol (in some instances, a bio-aerosol) sample and testing the aerosol for the presence (or absence) of pathogen or virus. The bio-aerosol can be, for example, from nasal or oral exhalation. As such, the sample collection and testing devices described herein enable rapid testing of an aerosol (in some instances, a bio-aerosol) sample and provide increased efficiency and decreased a cost and complexity. Moreover, the disclosed sample collection devices may minimize a possibility of contamination of the sample since both sample collection and testing can be performed in a single unit. In some embodiments, the samples can be collected using the sample collection devices described herein and sent to a lab for processing. Further, the disclosed sample collection device may be easily used by a user (e.g., a potential patient) without any prior training or professional help.

Some embodiments of the present disclosure relate to sample collection and testing devices that include a sample collection portion or device that can be attached to a sample testing portion or device to form an integrated sample collection and testing device.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a schematic front perspective view of one exemplary embodiment of a sample collection and testing device according to the present disclosure.

FIG. 2 is a schematic side view of the sample collection and testing device of FIG. 1.

FIG. 3A is a perspective schematic side view of the second portion of the sample collection device of FIG. 1.

FIG. 3B is a perspective schematic top view of the second portion of the sample collection device of FIG. 1.

FIG. 4A is a perspective schematic side view of the second portion of the sample collection device shown in FIGS. 3A and 3B approaching connection with the first portion to of the sample collection device to form the combined of the sample collection device of FIG. 1.

FIG. 4B is a perspective schematic bottom view of second portion of the sample collection device shown in FIGS. 3A and 3B in connection with the first portion of the sample collection device to form the sample collection device shown in FIG. 1.

FIG. 5 is an exploded schematic side view of the connected first and second portion shown in FIG. 4B.

FIG. 6 is a schematic side view of the three-dimensional porous sample collection device of FIG. 4B or 5.

FIG. 7A is perspective side view schematic of a sample collection device including an optional latching system.

FIG. 7B is an expanded view of a portion of the sample collection system of FIG. 7A.

FIG. 7C is a bottom perspective view schematic of the sample collection device of FIG. 7A.

FIG. 7D is an expanded view of the sample collection device of FIG. 7C along the longitudinal axis of the testing device.

FIG. 8 is perspective schematic view of an exemplary embodiment of an attachment mechanism including the latching system of FIGS. 7A-7D.

FIGS. 9A and 9B are, respectively, perspective and top view schematics of an exemplary pleating mechanism that can optionally be included in the sample collection devices of the present disclosure.

FIG. 10 is a side schematic view of a sample collection device including the pleating mechanism of FIGS. 9A and 9B and the latching system of FIGS. 7A-8.

FIG. 11 is a partial cut-away view of the sample collection device of FIG. 10.

FIG. 12 is a photograph of a pleated sample collection media formed by the plating mechanism of FIGS. 9A-11.

### DETAILED DESCRIPTION

Some embodiments of the present disclosure relate to sample collection and testing devices that include a sample collection portion or device that can be attached to a sample testing portion or device to form an integrated sample collection and testing device.

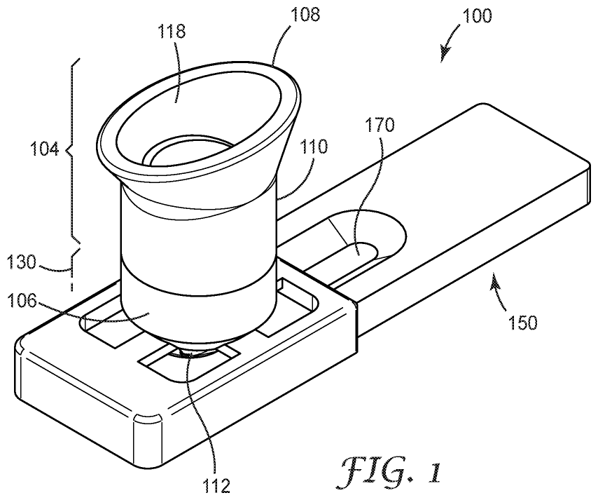

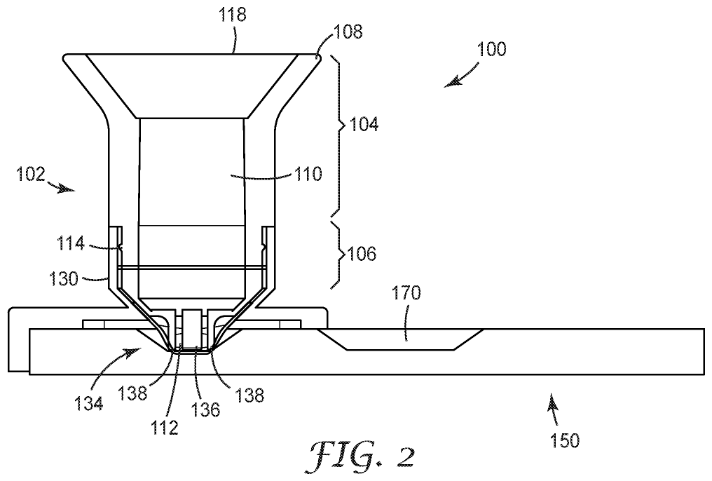

FIGS. 1 and 2 illustrate different views of an exemplary sample collection device 110 connected to an exemplary sample testing device 150 that together form an integrated sample collection and testing device 100. A user exhales into the mouthpiece or exhalation receipt portion 108 or the first portion 104 to introduce an exhalation airflow into the sample collection device 102. The user's exhalation can be nasal or oral exhalation. The exhalation airflow flows through a fluid channel in blow tube 110 and onto porous sample collection media 112. Viral or pathogen material is incident upon and adheres to or bonds with the porous sample collection media 112 to form a porous sample collection media loaded with exhaled material (referred to as a loaded sample collection media).

The user then attaches (either permanently or temporarily) the sample testing device 150 to the sample collection device 102 by, for example, snapping the two pieces together. In some embodiments, the sample collection device 102 and sample testing device 150 are connected to form the integrated sample collection and testing device 100 before the user exhales into the mouthpiece or exhalation receipt portion 108 or first portion 104. The user then introduces a fluid into the device (for example, by dropper or bottle) through one or more fluid inlet ports 118. In this implementation, the one or more fluid inlet ports 118 are the same holes or apertures forming the blow tube 110. The fluid introduced through the one or more fluid inlet ports 118 is incident upon the loaded porous collection media 112 and forms an eluent that moves from the loaded porous collection media 112 toward assay 160 in testing device 150. More specifically, the fluid travels through the loaded porous sample collection media 112 and carries viral and pathogen that was present on the loaded porous sample collection media 112 in the eluent toward the assay 160 (not shown) for testing. Optionally, the sample testing device 150 includes a visual indicator 170 informing the user of the presence or absence of viral or pathogenic material in the exhalation airflow the user provided into the sample collection device 102.

The two-part design of the sample collection and testing device 100 may facilitate disposability and/or reuse of one or more of the sample collection device 102 and/or the sample testing device 150. The two-part design also enables the user to use a variety of sample testing devices with, for example, differing assays to test for differing virus or pathogen presence.

Each of the portions of the sample collection and testing devices described herein and one implementation of which is shown as sample collection and testing device 100 are described in greater detail below.

### Sample Collection Devices.

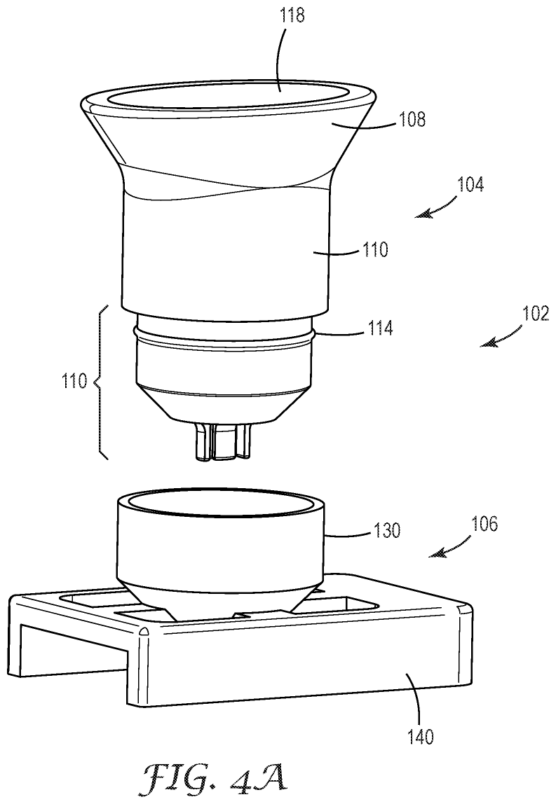

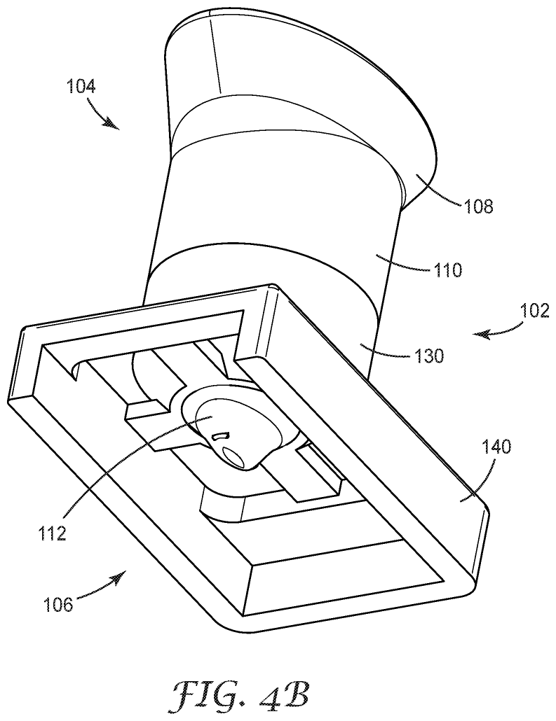

As shown in FIG. 4A, the sample collection device 102 includes a first portion 104 and a second portion 106. First and second portions 104 and 106 jointly form a housing including a fluid channel through which air and/or fluid (provided, for example, by exhalation of a user and called, for example, an exhalation airflow) entering through first portion 104 flows before exiting through second portion 106. As used herein, the term “fluid” may refer to a liquid or gas (i.e., air). First and second portions 104 and 106 are shown here as separate and attachable, but they may be integral (a single unit). Where the first and second portions 104 and 106 are attachable, they may be permanently attachment or temporarily attachable. In some embodiments, first portion 104 includes a mouthpiece or exhalation receipt portion 108 (optional). A user exhales into the mouthpiece or exhalation receipt portion 108 or the first portion 104 to introduce an exhalation airflow 110 into the sample collection device 102. The mouthpiece or exhalation receipt portion is configured to receive an exhalation airflow from one or more of the mouth or nose. For example, the mouthpiece or exhalation receipt portion can be breathed into by contact with or adjacency to the mouth or by contact with the nose/nostril or by contact with each individually or collectively. The exhalation airflow received by the mouthpiece or exhalation receipt portion can be one or both of oral exhalation or nasal exhalation. The term “mouthpiece.” where used herein, is meant to refer to an exhalation receipt portion that can receive oral or nasal exhalation of aerosol. The sample collection device 102 may be formed of a rigid material, such as plastic. In some embodiments, the first portion 104, the second portion 106, and/or mouthpiece or exhalation receipt portion 108 may be integral parts of the housing 102 or one or more of them may be separate parts that can be detached, attached, or reattached.

First portion 104 forms a blow tube 109 through which the exhalation airflow flows and through which fluid introduced through fluid inlet port 118 flows. The blow tube 109 includes an optional mouthpiece or exhalation receipt portion 108 at a first terminal end and an attachment mechanism 134 at a second terminal end.

The attachment mechanism 134 of FIG. 4A is merely exemplary and others can be used. The exemplary attachment mechanism 134 includes an interference bump 114 that hits the wall of the media holder 130 and one or more posts 136 and a plurality of fingers or contact elements 138 that engage with the media holder or receptacle 130 of second portion 106. As shown in FIG. 2, fingers or contact elements 138 have a generally L-shape that enables them to have some degree of vertical and/or horizontal flex, allowing them to act like springs and provide a degree of compliance to the system. In some embodiments, the fingers and/or pos flex in tangent to the plane to the assay test strip (in other words, they flex in the Z direction/along the Z axis). In some embodiments the compliance is along the blow tube 110 or fluid channel longitudinal axis. The one or more posts 136 can be more rigid than the fingers or contact elements 138. The post(s) 136 and finger elements 138 may hold the porous sample collection media adjacent to or in direct contact with the assay. In the specific implementation shown, the embodiment has four fingers or contact elements 138, but more or fewer may be used and still fall within the scope of the present application. The fingers or contact elements 138 also compensate for height variances in the system so that attachment of first and second portions 104 and 150 can be effected with ease by the user and without excessive force that could cause the porous sample collection media 112 to tear, thus possibly rendering the test ineffective.

The one or more fluid inlet ports 118 are disposed in fluid communication with the porous sample collection media 112. The one or more fluid inlet ports 118 may deliver the test fluid, via capillary action, to the porous sample collection media 112.

In some embodiments, the sample collection device 100 further includes a screen (not shown) disposed in the housing, blow tube 110, or mouthpiece or exhalation receipt portion 108 and upstream of the porous sample collection media 112 to catch solid material or debris (such as, for example, food particles) so that they are not incident on the porous sample collection media 112. In some embodiments, the screen includes one or more flow apertures therethrough. The exhalation airflow passes through a thickness of the screen. The screen at least partially occludes the fluid channel. In some cases, the screen may have a major plane (not shown) that is orthogonal to the direction of the exhalation airflow (not shown) passing through the thickness of the screen. The screen may be a non-woven layer configured to filter out larger particles from the exhalation airflow passing through the screen. In some cases, the screen may be a non-woven layer that does not have an electrostatic charge. In some embodiments, the screen does not capture significant amounts of viral or pathogen material and instead allows them to transmit through the screen. In some embodiments, the screen is made of or includes at least one of a plastic mesh, a woven net, a needle tacked fibrous web, a knitted mesh, an extruded net, and/or a carded or spunbond coverstock. In some embodiments, the screen does not catch or remove from the airflow particles having a size of less than 100 micrometers, or 75 micrometers, or 50 micrometers, or 25 micrometers, or 10 micrometers, or 5 micrometers.

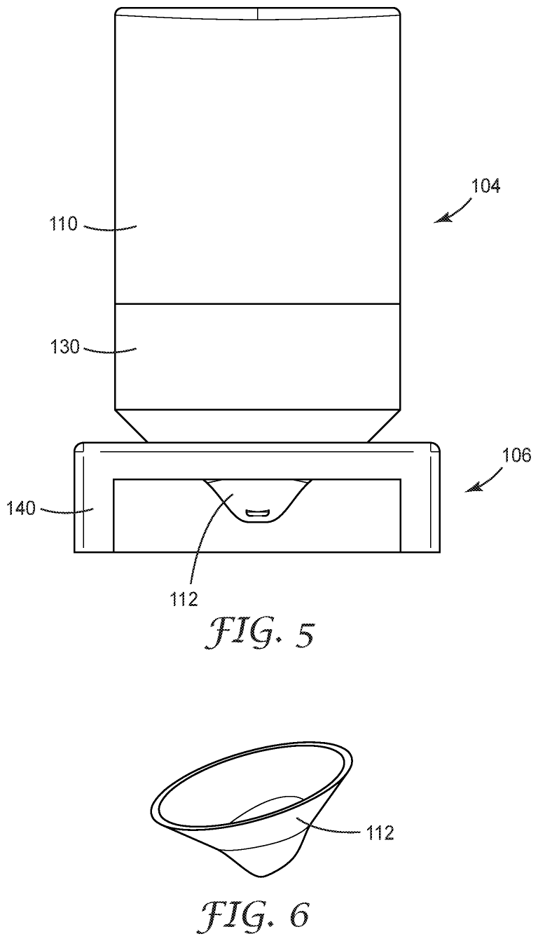

Second portion 106 includes (1) a sample media receptacle 130 into which porous sample collection media 112 is placed and held; and (2) an attachment mechanism 140 that attaches the sample collection device 102 to the sample testing device 150. In the exemplary implementation of FIGS. 1-7, the attachment mechanism 140 involves the user attaching the sample testing device 150 to the sample collection device 110 by, for example, snapping the two pieces to one another. Those of skill in the art will appreciate that other attachments mechanisms and processes can be used including, for example, sliding, screwing, clipping, or rotation. Sample media holder or receptacle 130 holds porous sample collection media 112 within the sample collection device 102 such that it is in fluid communication with the fluid channel and at least partially occludes the fluid channel.

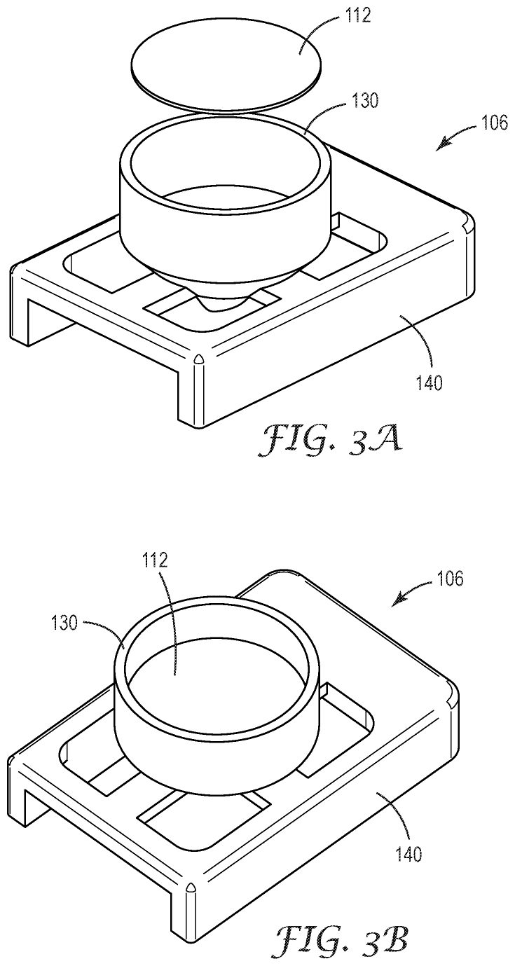

FIGS. 3A-4B show the process by which the two-dimensional porous sample collection media 112 is placed in and held within sample media receptacle 130 and how the two-dimensional porous sample collection media 112 becomes a three-dimensional porous sample collection media 112. As shown in FIG. 3A, a piece of porous sample collection media 112 is placed within sample media receptacle 130 on or adjacent to a sample media holder 132 (not shown) on which sample collection media 112 rests. Exemplary sample media holders 132 (not shown) include a lip or edge on the inner surface of sample media receptacle 130 or a screen on the bottom of sample media receptacle 130 on which porous sample collection media 112 rests. The sample media holders 132 preferably allow air to pass through them. FIG. 3B shows porous sample collection media 112 resting on sample media holders 132 (not shown) within sample media receptacle 130. In some embodiments, the porous sample collection media 112 is attached to the sample media holder 132 by an adhesive (for example, a pressure sensitive adhesive, a porous adhesive, a structured adhesive, a heat-activated adhesive, and/or a medical grade adhesive). The adhesive may be continuous or discontinuous. The adhesive may be patterned or continuous. In some embodiments, the porous sample collection media 112 is attached to the sample media holder 132 by hook and loop and/or 3M™ Dual Lock™ Reclosable Fasteners. In some embodiments, the porous sample collection media 112 is attached to the sample media holder 132 by a means of mechanical attachment, such as pins, stapling, tongue and groove connections, etc.

FIGS. 4A and 4B show how first portion 104 of the sample collection device 102 is attached or placed adjacent to second portion 106 of the sample collection device 102. Attachment of first and second portions 104 and 106 cause porous sample collection media 112 to change from a two-dimensional, flat shape or structure to a three-dimensional shape or structure. The specific three-dimensional shape or structure shown in FIGS. 4B and 6 is a frustoconicular shape or structure but those of skill in the art will understand that this shape can be changed depending on the number, shape, and placement of post(s) 136 and fingers or contact elements 138 and/or the shape of the media receptacle 130. Further, those of skill in the art will appreciate that while the two-dimensional porous sample collection media 112 is shown as circular, it can be any desired shape including, for example, at least one of circular, oval, elliptical, square, rectangular, triangular, trapezoidal, pentagonal, hexagonal, heptangular, or octagonal.

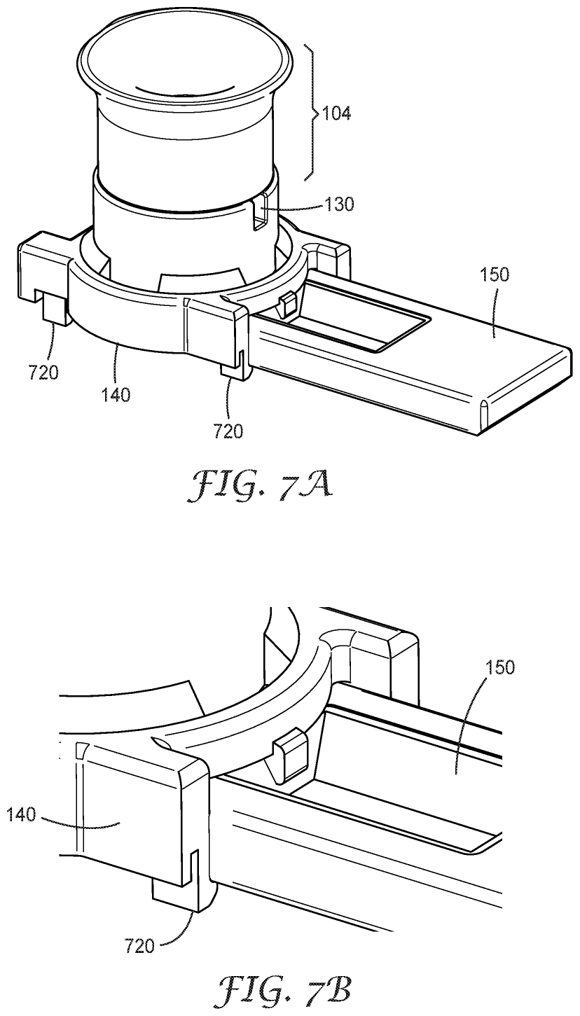

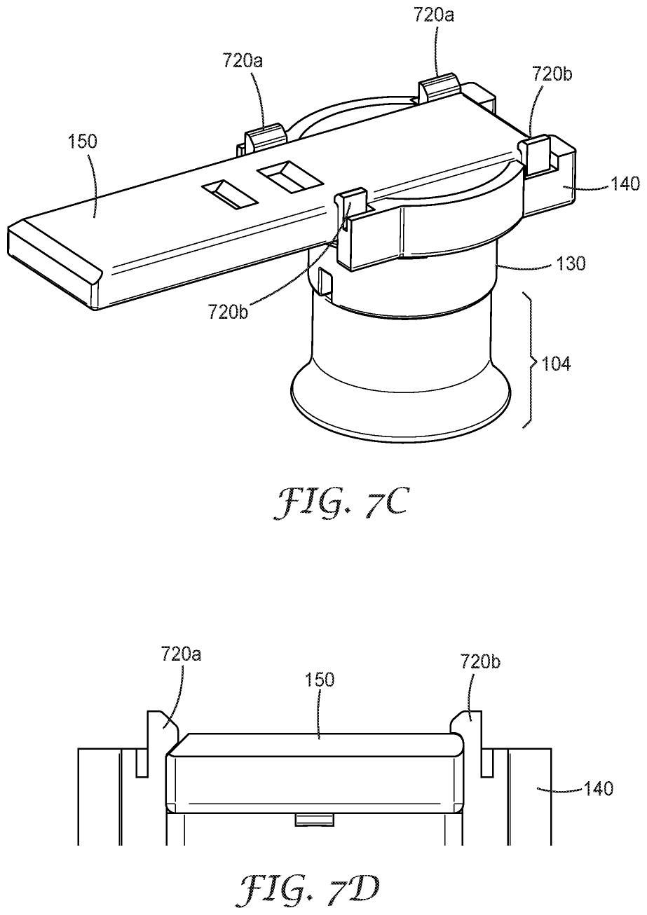

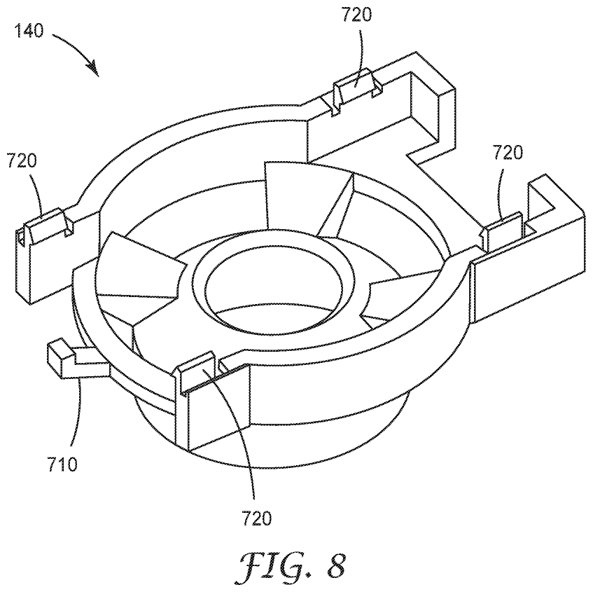

FIGS. 7A-7D and 8 shown an optional, exemplary latching system that that may be included in attachment mechanism 140. The latching system includes two features, either or both of which may optionally be present. The first feature of the optional latching system is a front latch or clip 710 in/on the attachment mechanism that attaches to the sample testing device 150. Front latch or clip 710 prevents the sample testing device 150 from sliding or moving within the sample collection and testing device.

The second feature is a set of side latches or clips 720 that securely hold the sample testing device 150 in position. The implementation shown has 4 side latches or clips 720 and two different types of latches. Latch 720a is a chamfered latch with a generally 45 degree angle. Latch 720b is a radiused latch. The type, number, placement, or orientation of latches of clips 720 is merely exemplary, and any desired type, number, placement, or orientation of latches of clips 720 may be used. The latches may all be the same or may differ from one another. In addition to holding the sample testing device 150 securely in place, latches or clips 720 also ensure correct positioning, alignment, and/or attachment of the sample testing device 150 to the breath capture tube. Together, these attachment features hold the sample testing device 150 securely in position in all planes and directions.

In some embodiments, the porous sample collection media 112 may be replaceable and changed out by a user, as desired. For example, a user may exhale, via the first portion 104, into the sample collection device 102 and load the porous sample collection media 112 with a sample of the exhalation airflow to form a loaded porous sample collection media. The user may then test the loaded porous sample collection media 112 as described herein. After conducting the test, the user may dispose of the loaded and tested porous sample collection media 112 and may then replace the loaded porous sample collection media 112 with an unloaded porous sample collection media 112 using the process described above.

### Sample Testing Devices

Sample testing device 150 attaches to the sample collection device 102 by, for example, snapping the two pieces together. Sample testing device 150 includes the assay 160 (not shown) that tests the eluent (and thus, by proxy, the exhalation airflow) for the presence or absence of virus or pathogen. The assay 160 is adjacent to or in direct contact with the porous sample collection media 112 such that the eluent flowing, for example, by capillary action, from the porous sample collection media 112 is incident on the assay and carries viral and pathogen that was present on the loaded porous sample collection media 112 in the eluent toward the assay 160 (not shown) for testing. In some embodiments, the assay is in direct physical contact with the porous sample collection media to enable receipt an eluent from the porous sample collection media. In some embodiments, the assay is adjacent to, but not in direct physical contact with, the porous sample collection media to enable receipt an eluent from the porous sample collection media.

The assay 160 is configured to receive a fluid from the porous sample collection media 112. Specifically, the assay 160 is configured to receive the test fluid from the porous sample collection media 112. Therefore, the assay 160 is disposed in fluid communication with the porous sample collection media 112. Generally, the eluent is collected and tested by the assay 150. In some embodiments, the assay 150 detects virus or pathogen presence in the exhalation airflow 110 and/or the test fluid. In other words, the assay 150 first collects the eluent and then detects virus or pathogen presence in the eluent.

Assay 160 qualitatively assesses or quantitatively measures the presence, amount, and/or functional activity of an analyte on the sample collection media 112. The analyte can be a drug, biochemical substance, chemical element or compound, or cell in an organism or organic sample. Exemplary biological assays include, for example, PCR-ELISA or Fluorescence. Assay 160 can detect a molecule, often in low concentrations, that is a marker of disease or risk in the aerosol (in some instances, a bio-aerosol) sample taken from the user/patient.

In some embodiments, the mechanism that holds the sample collection and sample testing devices adjacent to one another restricts movement of the sample collection device relative to the assay. In some embodiments, the restricted movement is perpendicular to the plane of the assay test strip (in other words, no movement in the Z direction/along the Z axis). In some embodiments, the restricted movement is parallel to the plane of the assay test strip (in other words, no movement in the X or Y directions/along the X or Y axis).

The sample collection devices of the present disclosure can be used to collect droplets, aerosols or particulates to make a sample of material collected from a subject, group, or area. To collect the sample, the surface charge is reduced by wetting the surface. This can be accomplished by surfactants, wetting agents, addition of surface energy matched solvents, or mechanical means. Once the sample has been wetted, the collected material can be conveyed within the fluid for testing.

In some embodiments, the assay is a lateral flow assay or a vertical flow assay. In general, lateral flow assays or vertical flow assays are paper-based platforms for the detection and quantification of analytes in complex mixtures, where a sample is placed on a test device and the results are displayed within 5-30 mins. Low development costs and ease of production of lateral flow assays have resulted in the expansion of its applications to multiple fields in which rapid tests are required. Lateral flow assay-based tests are widely used in hospitals, physician's offices and clinical laboratories for the qualitative and quantitative detection of specific antigens and antibodies, as well as products of gene amplification. A variety of biological samples can be tested using assays.

Optionally, the sample testing device 150 also includes a visual indicator 170 informing the user of the presence or absence of viral or pathogenic material in the exhalation airflow the user provided into the sample collection device 102. The visual indicator can include, for example, letters, colors, words, shapes, or lines that indicate the presence (or absence) of viral or pathogenic material. The sample testing device 150 may include a display window 154 configured to allow visual inspection of at least a portion of the assay 160. Specifically, a user can see the test results on the assay 160, via the display window, and can get an indication of presence or absence of pathogens in the eluent and/or the test fluid.

Optionally, the sample testing device 150 may include a barrier (not shown) disposed between the fluid channel and the assay 160. The barrier may be configured to prevent direct fluid communication between the exhalation airflow and the assay 160 in instances where the sample collection device 102 and the sample testing device 150 are attached before the user exhales into or introduces an exhalation airflow into the sample collection device 102. The barrier may cover or enclose an entire area of the assay 160 to act as a layer between the fluid channel and the assay 160.

Test Fluid. In some embodiments, the test fluid is an aqueous solution including a surfactant. In some embodiments, the test fluid may be an aqueous buffer solution. In some embodiments, the test fluid may be a saline solution. In some embodiments, the test fluid may be a saline solution including a surfactant. In some embodiments, the test fluid may be a saline solution including from about 0.5% to about 2% surfactant by weight.

Media. In some embodiments, the porous sample collection media 112 includes a nonwoven material. In some embodiments, the nonwoven material (e.g., a nonwoven filtration material) has an electrostatic charge. In some embodiments, the porous sample collection media 112 includes a nonwoven filtration material having an electrostatic charge configured to filter pathogens from the exhalation airflow. In some embodiments, the nonwoven filtration material is hydrophobic. Thus, the hydrophobic nonwoven filtration material may be configured to filter pathogens from the exhalation airflow. In some cases, the porous sample collection media 112 may have a thickness (orthogonal to the major plane 114) in a range from 200 micrometers (mm) to 1000 mm, or from 250 mm to 750 mm.

As used herein, the term “hydrophobic” refers to a material having a water contact angle of 90 degrees or greater, or from about 90 degrees to about 170 degrees, or from about 100 degrees to about 150 degrees. Water contact angle is measured using ASTM D5727-1997 Standard test method for surface wettability and absorbency of sheeted material using an automated contact angle tester.

In some cases, the porous sample collection media 112 may be formed of a polymeric material. In some cases, the porous sample collection media 112 may be formed of a polyolefin. In some embodiments, the porous sample collection media 112 may be formed of polypropylene. In some embodiments, the porous sample collection media 112 may be formed of a polylactide (PLA) such as, for example, 6100D from NatureWorks LLC15305 Minnetonka Blvd Minnetonka. MN 55345. Exemplary nonwoven materials for use in or as the porous sample collection media 112 include, for example, those described in U.S. Pat. Nos. 7,947,142; 8,162,153; 9,139,940; and 10,273,612, all of which are incorporated herein in their entirety.

In some embodiments, the nonwoven media is pleated. The sample collection media itself may be pleated.

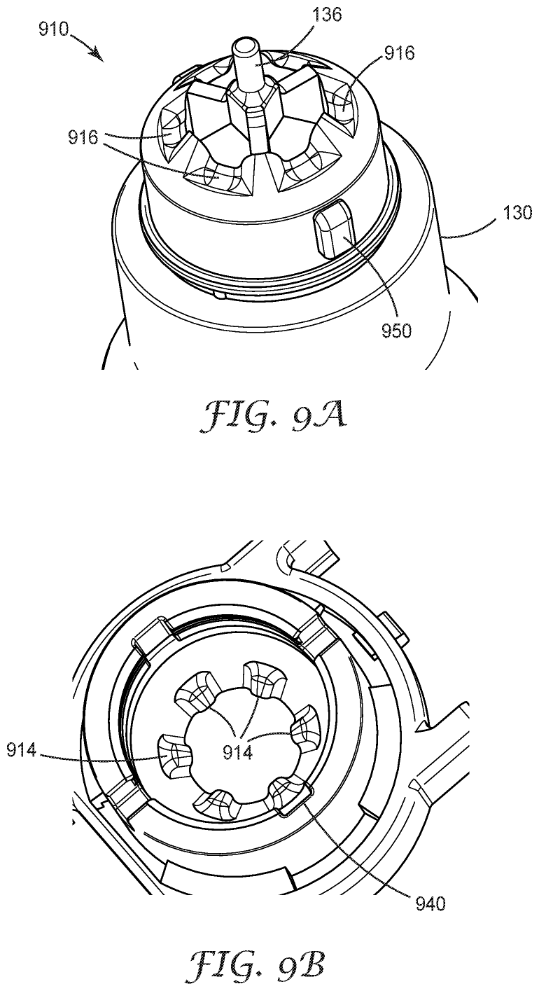

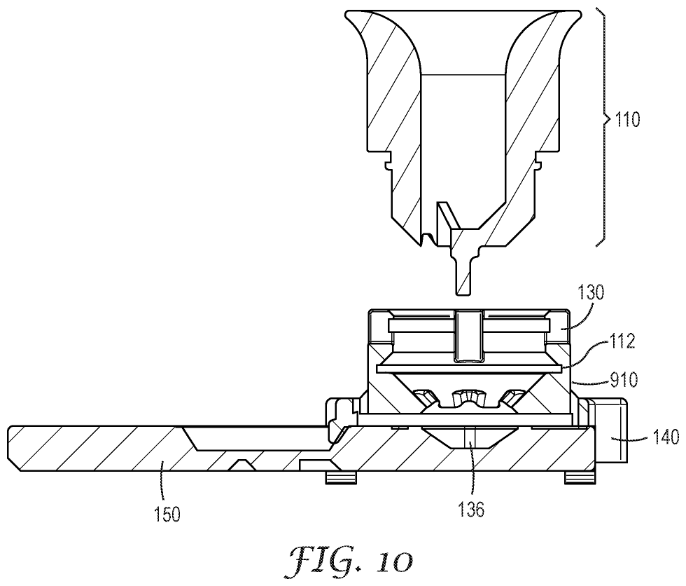

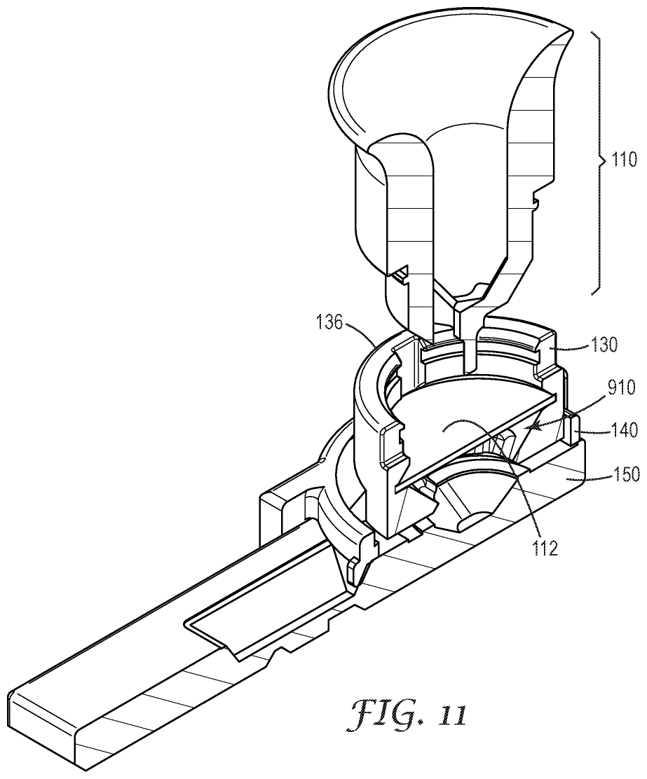

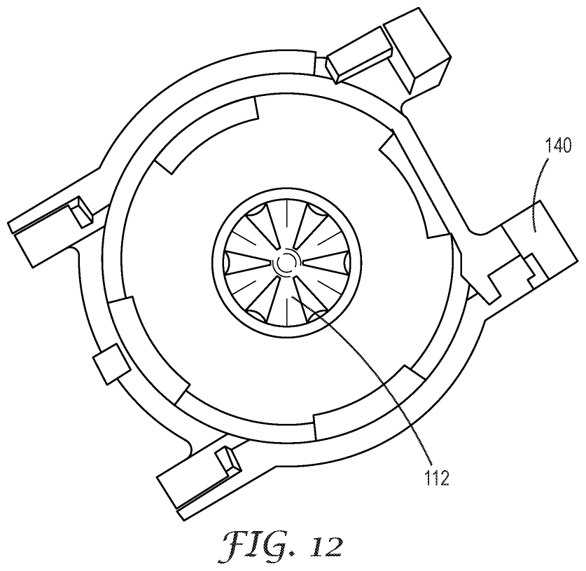

Alternatively, the sample collection device may include a pleating mechanism that creates a pleating pattern or shape in an initially flat or relatively flat sample collection media 112. One exemplary pleating mechanism is shown in FIGS. 9-12. Pleating mechanism 910 includes a plurality of pleating features that, in this implementation, are ridges 914 and recesses 916. Ridges 136 are in the media holder 130 and recessed 916 are in the attachment mechanism 140 (although this is merely exemplary and the two may be reversed). As shown in FIGS. 10 and 11, when a relatively flat piece of sample collection media is placed between the media holder 130 and the attachment mechanism 140 and the two pieces 130 and 140 are tightly pressed together, peaks and valleys (and thus pleats) are formed in the in the sample collection media 112. This occurs when post 136 pushes against the sample collection media 112, causing the sample collection media 112 to take the shape of the pleating features (e.g.: ridges 914 and recesses 916. Post 136 may also hold the sample collection media adjacent to the testing device 150.

In some embodiments, the media holder 130 includes a pre-staging area that holds the sample collection media 112 proximate to the pleating mechanism until the attachment mechanism is attached or pushed adjacent thereto. This is shown in FIGS. 10 and 11.

In some embodiments, the media holder 130 and attachment mechanism 140 lock into place which holds the sample collection media 112 in the pleated format. The specific pleating pattern or shape of the pleating features shown in FIGS. 9-12 is a generally frustoconical, pleated shape and/or pattern, but any desired pattern and shape may be used. Further, the specific number of ridges 914 and recesses 916 shown in FIGS. 9A-11 is exemplary and can be changed as desired. For example, the number, size, and spacing of the pleating features may vary based on the size sample collection media and/or the height and shape of the cone or cylinder shape.

FIG. 12 is a photograph of the sample collection media 112 in its pleated form.

The specific embodiment of FIGS. 9A-11 also includes an optional alignment slot 940 and matable alignment tab 950 that align and mate when the media holder 130 and attachment mechanism 140 are attached to one another. Alignment tab 950 ensures that the media holder 130 and attachment mechanism are mated or attached such that the ridges 914 and recesses 916 align.

In some embodiments, the pleat frequency is between about 1 pleat per 0.6 cm of media and about 1 pleat per 2 mm of media. In some embodiments, the pleat height is between about 2 mm and about 4 mm.

Pleated sample collection media offers certain advantages, including increased surface area and improved alignment of the sample collection media within the sample collection device. The pleating mechanism, specifically, eliminated any slop in the sample collection media by forming the pleating. This results in better sample collection media alignment and thus better performance of the sample collection device.

Pressing Element. In some cases, the sample collection device 100 may include a pressing element (not shown) that is configured to apply pressure onto the loaded porous sample collection media 112. The pressing element may force a remaining test fluid out of the loaded porous sample collection media 112 for collection and testing.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

The term “substantially” as used here has the same meaning as “significantly,” and can be understood to modify the term that follows by at least about 90%, at least about 95%, or at least about 98%.

The term “about” is used here in conjunction with numeric values to include normal variations in measurements as expected by persons skilled in the art and is understood have the same meaning as “approximately” and to cover a typical margin of error, such as +5% of the stated value.

Terms such as “a,” “an,” and “the” are not intended to refer to only a singular entity but include the general class of which a specific example may be used for illustration.

The terms “a.” “an.” and “the” are used interchangeably with the term “at least one.” The phrases “at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have”, “having”, “include”, “including”, “comprise”, “comprising” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method or the like, means that the components of the composition, product, method or the like are limited to the enumerated components and any other components that do not materially affect the basic and novel characteristic(s) of the composition, product, method or the like.

Any direction referred to here, such as “front,” “back,” “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

The terms “downstream” and “upstream” refer to a relative position based on a direction of exhalation airflow through the device.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A sample collection device comprising:
a housing extending from a first portion to a second portion, the housing defining a fluid channel from the first portion to the second portion, wherein the first portion is configured to receive an exhalation airflow and the second portion is configured to be an air outlet portion;
a porous sample collection media comprising nonwoven filtration material and having a frustoconical shape and disposed within the fluid channel; and
a fluid inlet port through which fluid may be introduced into the housing such that the fluid is incident on the porous sample collection media.

2. The sample collection device of claim 1, further comprising:
a sample testing device comprising an assay attached to the sample collection device such that the assay and porous sample collection media are adjacent to or in direct contact with one another.

3. The sample collection device of claim 2, wherein the sample collection device and the sample testing device are attachable to and/or detachable from one another by at least one of snapping, sliding, screwing, clipping, or rotation.

4-5. (canceled)

6. The sample collection device of claim 1, wherein the porous sample collection media comprising nonwoven material has an electrostatic charge.

7-8. (canceled)

9. The sample collection device of claim 1, wherein the assay is capable of detecting virus or pathogen presence in the exhalation airflow and/or an eluent.

10. The sample collection device of claim 1, wherein the assay is a lateral flow assay or a vertical flow assay.

11. (canceled)

12. The sample collection device of claim 1, where in the second portion comprises a post, a plurality of fingers, a plurality of contact elements, or a combination thereof, that are in contact with the sample collection media and hold the sample collection media in contact with the assay.

13. The sample collection device of claim 12, wherein the fingers or contact elements are non-rigid and have compliance along longitudinal axis of the fluid channel.

14. A method for testing an exhalation airflow for the presence or absence of a virus or pathogen, the method comprising:
exhaling into a sample collection device comprising:
a housing having a fluid channel;
a frustoconical-shaped porous sample collection media disposed within the fluid channel,
the porous sample collection media comprising nonwoven filtration material,
wherein the exhalation airflow flows through the porous sample collection media such that the exhalation airflow is incident upon the porous sample collection media and forms a loaded porous sample collection media;
placing a liquid in contact with the loaded porous sample media such that the liquid contacts and passes through the loaded porous sample media to form an eluent that is incident upon an assay; and
testing the eluent, by the assay, for presence of a virus or pathogen in the eluent.

15. (canceled)

16. The method of claim 1, wherein the porous sample collection media comprising nonwoven filtration material having has an electrostatic charge.

17. (canceled)

18. The method of claim 1, wherein the test fluid is at least one of an aqueous fluid, an aqueous buffer solution, an aqueous fluid including a surfactant, a saline solution, or a saline solution including a surfactant.

19. The method of claim 1, wherein the assay is a lateral flow assay or a vertical flow assay.

20. A method of forming a sample collection device, comprising:
attaching a first portion and a second portion of a sample collection device to one another,
the first portion comprising a fluid inlet portion and fluid channel through which fluid may flow;
the second portion comprising a media receptacle comprising a sample media holder and a porous sample collection media adjacent to the sample media holder;
wherein attaching the first portion and second portion to one another to causes the porous sample collection media to change from a two-dimensional, flat shape or structure to a three-dimensional shape or structure.

21. The method of claim 20, wherein the three-dimensional shape or structure created by the inverse of the shape or structure provided by at least one of the media receptacle, post(s), and/or fingers or contact elements.

22. The method of claim 20, wherein the porous sample collection media shape is frustoconical.

23. The method of claim 20, wherein the porous sample collection media comprises a nonwoven material having an electrostatic charge.

24-25. (canceled)

26. The method of claim 20, further comprising attaching the sample collection device to a lateral flow assay or a vertical flow assay.

27. The method of claim 20, wherein the two-dimensional porous sample collection media has a shape of at least one of circular, oval, elliptical, square, rectangular, triangular, trapezoidal, pentagonal, hexagonal, heptangular, or octagonal.

28. A sample collection and testing device comprising:
a sample collection portion comprising:
a housing formed by a first portion and a second portion, the housing defining a fluid channel extending from the first portion to the second portion;
the first portion being configured to receive an exhalation airflow; and
the second portion being configured to be an air outlet portion and to attach a sample testing portion to the sample collection portion by means of one or more posts and fingers that cooperatively engage with the sample testing portion to hold the sample collection portion and the sample testing portion in engagement; the post and fingers also engaging with the porous sample collection media to hold it in place and/or alter its shape;
a porous sample collection media disposed within the fluid channel and in direct contact with the post and/or fingers; and
a fluid inlet port through which fluid may be introduced into the housing such that the fluid is incident on the porous sample collection media; and
a sample testing portion that can be attached to the sample collection portion and including: an assay that can be attached to the sample collection device such that the assay and porous sample collection media are adjacent to or in direct contact with one another.

29. (canceled)

30. The sample collection device of claim 1, wherein the sample collection media is pleated.

31-33. (canceled)
